(19) United States
(12) Patent Application Publication
(10) Pub. No.: US 2018/0103880 A1
(43) Pub. Date: Apr. 19, 2018

(54) BLOOD GLUCOSE MONITORING DEVICE AND MONITORING METHOD THEREOF

(71) Applicant: BOE TECHNOLOGY GROUP CO., LTD., Beijing (CN)

(72) Inventor: Hailan JIN, Beijing (CN)

(73) Assignee: BOE TECHNOLOGY GROUP CO., LTD., Beijing (CN)

(21) Appl. No.: 15/539,116

(22) PCT Filed: Jan. 4, 2017
(86) PCT No.: PCT/CN2017/070164

§ 371 Date: Jun. 22, 2017

(30) Foreign Application Priority Data
Apr. 5, 2016 (CN) 201610207628.8

(51) Int. Cl. A61B 5/145; A61B 5/00

(52) U.S. Cl. CPC A61B 5/14532; A61B 5/1455; A61B 5/0028; A61B 5/14507

(57) ABSTRACT

Embodiments of the present disclosure provide a blood glucose monitoring device and a monitoring method thereof, which can reduce the influence of the oral environment on the measurement accuracy. The blood glucose monitoring device comprises: a housing; a micro-current stimulation module connected to two electrodes and generating a current, the two electrodes being located on a surface of the housing and the current stimulating the parotid gland of a user through the two electrodes to produce fresh saliva; a saliva glucose acquisition module configured to acquire a glucose concentration in the saliva; a control processing module configured to control the micro-current stimulation module to work and to obtain a blood glucose concentration value from the glucose concentration.

### RELATED APPLICATION

The present application is the U.S. national phase entry of PCT/CN2017/070164, with an international filing date of Jan. 4, 2017, which claims the benefit of Chinese Patent Application No. 201610207628.8, filed on Apr. 5, 2016, the entire disclosures of which are incorporated herein by reference.

### TECHNICAL FIELD

The present disclosure relates to the technical field of blood glucose monitoring, in particular to a blood glucose monitoring device and a monitoring method thereof.

### BACKGROUND

With the significant decrease in labor intensity and changes in eating habits of modern people, the incidence of diabetes is rising rapidly. The impacts caused by diabetes and its complications have become one of the serious public health problems that all countries in the world have to face.

At present, self-monitoring of blood glucose is a widely and internationally recognized diabetes management approach. But self-monitoring of blood glucose requires a patient to detect the blood glucose 5-6 times each day, which is very time-consuming and labor-consuming.

### SUMMARY

According to one aspect of the present disclosure, a blood glucose monitoring device is provided, which comprises: a housing; a micro-current stimulation module connected to two electrodes and generating a current, the two electrodes being located on a surface of the housing and the current stimulating the parotid gland of a user through the two electrodes to produce fresh saliva; a saliva glucose acquisition module configured to acquire a glucose concentration in the saliva; a control processing module configured to control the micro-current stimulation module to work and to obtain a blood glucose concentration value from the glucose concentration.

Optionally, said blood glucose monitoring device further comprises a display module configured to display the blood glucose concentration value obtained by the control processing module.

Optionally, said blood glucose monitoring device further comprises a wireless communication module through which the control processing module communicates with an external terminal.

Optionally, a current intensity control module is integrated in the control processing module to control intensity of current generated by the micro-current stimulation module. As an example, said blood glucose monitoring device further comprises a current intensity regulation button operated to control current intensity of the micro-current stimulation module through the current intensity control module; the current intensity regulation button may be provided on a surface of the housing. Or, in the case where said display module has a touch function, said control processing module can also be used for controlling the display module to display current intensity levels and controlling the intensity of current generated by the micro-current stimulation module using the current intensity control module according to a current intensity level selected by the user.

Optionally, said blood glucose monitoring device further comprises a test paper box detachably secured to the housing.

Optionally, the test paper box comprises a box body, an elastic component provided at the bottom of the box body and a push rod provided at the top of the box body; wherein a test paper outlet is provided on a side of the box body at a position close to the top; the push rod is disposed opposite to the test paper outlet, and the push rod extends from inside of the box body to outside of the box body.

Optionally, a portion of the push rod extending to the outside of the box body is located within the housing and is connected to the control processing module; and wherein the blood glucose monitoring device further comprises a test paper control button operated to control the push rod by the control processing module so as to push the test paper out of the test paper box. Or, in the case where the display module has a touch function, the control processing module is further used for controlling the display module to display the test paper control button, and controlling the push rod to push out the test paper according to the user's selection of the test paper control button.

Optionally, the blood glucose monitoring device further comprises a power supply module which supplies power to the blood glucose monitoring device. A battery placement area may also be provided in the housing to place a battery for supplying power to the power supply module; and/or a power supply interface is also provided on the housing, through which an external power supply supplies power to the power supply module.

Optionally, the blood glucose monitoring device further comprises an alarm module for providing an alarm signal under the condition that the control processing module determines that the blood glucose concentration value is greater than a preset threshold value.

Optionally, the housing is made of a flexible material.

According to another aspect of the present disclosure, there is provided a monitoring method for the above-mentioned blood glucose monitoring device, comprising: after receiving a blood glucose monitoring instruction, stimulating a user's parotid gland by a micro-current to produce fresh saliva; obtaining a glucose concentration from the fresh saliva; and converting the glucose concentration into a blood glucose concentration value.

Optionally, said method further comprises displaying the blood glucose concentration value.

Optionally, said monitoring method further comprises sending an alarm signal when the blood glucose concentration value is greater than a preset threshold value.

By means of the blood glucose monitoring device and monitoring method thereof provided in embodiments of the present disclosure, the micro-current stimulation module can be used first to stimulate the user to produce fresh saliva and then the saliva glucose acquisition module is used to detect the glucose concentration in the fresh saliva; after processing the acquired glucose concentration by the control processing module, the blood glucose concentration value can be obtained, then the blood glucose concentration value can be transmitted to an external device through the wireless communication module. In this way, influences from food residues or other substances in the mouth can be eliminated, and influence to the accuracy of measurement by the oral environment can be reduced, thereby improving monitoring accuracy and reliability, meanwhile, the problem that the test cannot be carried out when the user has a dry mouth without saliva is also solved.

### BRIEF DESCRIPTION OF THE DRAWINGS

In order to more clearly illustrate the embodiments of the present disclosure, the drawings that are to be used in the descriptions of the embodiments will be briefly introduced below. The drawings in the following description are merely some embodiments of the present disclosure, and other drawings can be obtained by those skilled in the art from these drawings without using inventive skills.

FIG. 1 is a first schematic diagram of an internal structure of a blood glucose monitoring device according to an embodiment of the present disclosure;

FIG. 2 is a second schematic diagram of an internal structure of a blood glucose monitoring device according to an embodiment of the present disclosure;

FIG. 3 is a first schematic diagram of a housing surface of a blood glucose monitoring device according to an embodiment of the present disclosure;

FIG. 4 is a third schematic diagram of an internal structure of a blood glucose monitoring device according to an embodiment of the present disclosure;

FIG. 5 is a second schematic diagram of a housing surface of a blood glucose monitoring device according to an embodiment of the present disclosure;

FIG. 6 is a first schematic diagram of a blood glucose monitoring device comprising a test paper box according to an embodiment of the present disclosure;

FIG. 7 is a schematic diagram of the structure of a test paper box according to an embodiment of the present disclosure;

FIG. 8 is a second schematic diagram of a blood glucose monitoring device comprising a test paper box according to an embodiment of the present disclosure;

FIG. 9 is a schematic diagram of a blood glucose monitoring device comprising a battery placement area according to an embodiment of the present disclosure;

FIG. 10 is a schematic diagram of a blood glucose monitoring device comprising a power supply interface according to an embodiment of the present disclosure;

FIG. 11 is a schematic diagram of a blood glucose monitoring device comprising a battery placement area and a power supply interface according to an embodiment of the present disclosure;

FIG. 12 is an internal structural diagram of a blood glucose monitoring device comprising an alarm module according to an embodiment of the present disclosure;

FIG. 13 is a flow chart of a monitoring method according to an embodiment of the present disclosure.

### DETAILED DESCRIPTION

### REFERENCE NUMERALS

10-housing; 101-opening; 102-switch; 103-current intensity regulation button; 104-test paper control button; 20-micro-current stimulation module; 201-first electrode; 202-second electrode; 30-saliva glucose acquisition module; 40-control processing module; 401-current intensity control module; 50-wireless communication module; 60-display module; 70-test paper box; 701-box body; 702-elastic component; 703-push rod; 704-test paper outlet; 705-bearing tray 80-test paper; 90-battery placement area; 100-power supply interface; 110-alarm module.

Technical solutions in the embodiments of the present disclosure will be now described clearly and completely with reference to the drawings. But obviously, the described embodiments are only some, instead of all, of the embodiments. All other embodiments that can be obtained by those ordinarily skilled in the art on the basis of the embodiments in the present disclosure without using any inventive skill shall fall into the protection scope of the present disclosure.

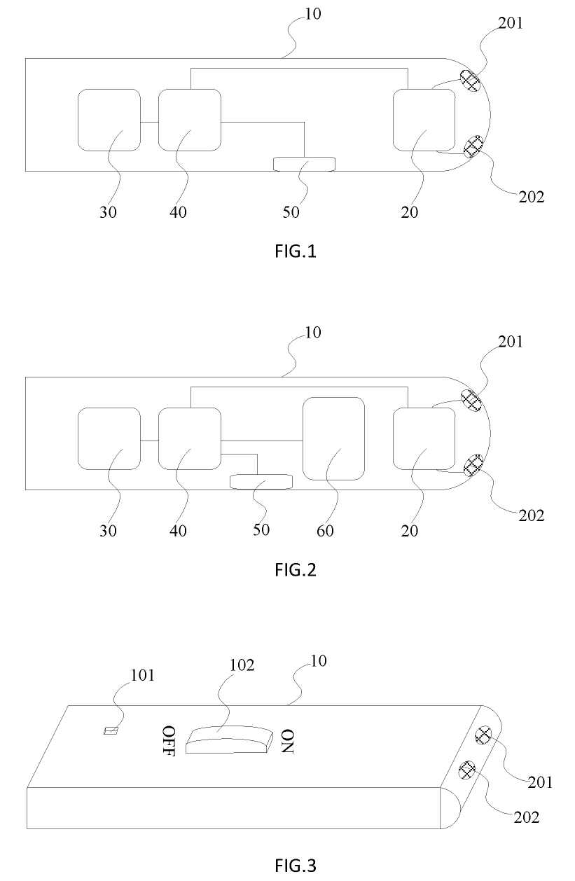

An embodiment of the present disclosure provides a blood glucose monitoring device, which, as shown in FIGS. 1-2, comprises a housing 10, a micro-current stimulation module 20, a saliva glucose acquisition module 30, and a control processing module 40. The blood glucose monitoring device may further comprise a wireless communication module 50.

The micro-current stimulation module 20 is connected to two electrodes, i.e. a first electrode 201 and a second electrode 202. Said two electrodes are on a surface of the housing 10, and current generated by the micro-current stimulation module stimulates the parotid gland of a user to produce fresh saliva.

The saliva glucose acquisition module 30 can be used for acquiring a glucose concentration in the saliva.

The control processing module 40 can be used for obtaining a blood glucose concentration value from the glucose concentration acquired by the saliva glucose acquisition module 30; and controlling the micro-current stimulation module 20 to work.

The control processing module 40 can communicate with an external terminal via the wireless communication module 50.

It shall be noted that one or more of said micro-current stimulation module, saliva glucose acquisition module and control processing module can be implemented as a processor, a component of an integrated circuit of a system-on-chip, an application specific integrated circuit (ASIC), a field programmable gate array (FPGA), a complex programmable logic device (CPLD), etc.

In the embodiment of the present disclosure, the current generated by the micro-current stimulation module 20 is a micro-current which can be a pulse current or a constant current. The intensity of the micro-current can be at a level of microampere (μA), for example, within a range of 1-500 μA.

When the blood glucose monitoring device of the present disclosure is used, the two electrodes, i.e., the first electrode 201 and the second electrode 202, can be brought into contact with the skin at the parotid gland, and the micro-current generated by the micro-current stimulation module 20 stimulates the parotid gland to produce fresh saliva. One of the first electrode 201 and the second electrode 202 is an anode and the other is a cathode.

The saliva glucose acquisition module 30 can, for example, use the test paper to obtain a glucose concentration in the saliva. The glucose concentration herein can be represented by current or be a glucose concentration value converted from the current value. As shown in FIG. 3, an opening 101 may be provided on the housing 10 of the blood glucose monitoring device, through which the test paper can be inserted into the housing 10 to be in contact with the saliva glucose acquisition module 30.

The above-mentioned test paper may detect glucose in saliva by means of an electrochemical enzyme detection method. The saliva glucose acquisition module 30 obtains a current value representing the glucose concentration by an electrode provided on the test paper.

As an example, the electrochemical enzyme method can be based on the reaction of glucose with glucose oxidase (GOD) and potassium ferricyanide.

The glucose in the saliva is catalytically oxidized to gluconic acid by the GOD on the test paper, and GOD is converted to its reduced state at the same time. That is, there is the following reaction formula: glucose+GOD(OX)→gluconic acid+GOD_{(RED)}; subscripts OX and RED represent the oxidation state and the reduced state, respectively.

Then, the reduced GOD reduces the ferricyanide ions in the potassium ferricyanide to ferrocyanide ions, which can be reflected by the reaction formula of DOD_{(RED)}+2M_{(OX)}→GOD_{(OX)}+2M_{(RED)}+2H⁺; M_{(OX)} and M_{(RED)} represent the ferricyanide ion and ferrocyanide ion, respectively.

When a certain voltage is applied to the test paper, the ferrocyanide ions are reduced to ferricyanide ions and a current is generated. That is, the following reaction formula can be used: 2M_{(RED)}→2M_{(OX)}+2e⁻; e⁻ represents electrons. The more the number of free electrons generated, the greater the current, and the current is proportional to the glucose concentration.

The control processing module 40 can, on the one hand, convert the glucose concentration acquired by the saliva glucose acquisition module 30 into a blood glucose concentration value. The blood glucose concentration y has a linear relationship with the glucose concentration x, i.e., y=a×x+b, wherein a and b can be pre-acquired and stored. On the other hand, the control processing module 40 can control the micro-current stimulation module 20 to work. In order to enable the blood glucose monitoring device to meet the needs of different users, for example, as shown in FIG. 3, a switch 102 may be provided on the surface of the housing 10. The switch 102 may normally be put in an ON state by default, so that when using the blood glucose monitoring device, the control processing module 40 controls the micro-current stimulation module 20 to work. When the user does not need the micro-current stimulation module 20 to generate stimulation current, the switch may be turned to an OFF state, so that the control processing module 40 does not control the micro-current stimulation module 20 to work.

The wireless communication module 50 may be Bluetooth, wifi, or the like. Through the wireless communication module 50, the control processing module 40 can send the blood glucose concentration value to an external device, which can be a mobile phone, a computer or a server.

In this case, the blood glucose concentration value obtained from each measurement can be stored by the peripheral device for easy management.

It is to be noted that since the housing 10 needs to be in contact with the human body, the material thereof should be non-toxic and wear resistant.

An embodiment of the present disclosure provides a blood glucose monitoring device which can use the micro-current stimulation module 20 to stimulate the user's parotid gland to produce fresh saliva. Thus influences from food residues or other substances in the mouth can be eliminated, and influence to the accuracy of measurement by the oral environment can be reduced, thereby improving monitoring accuracy and reliability, meanwhile, the problem that the test cannot be carried out when the user has a dry mouth without saliva is also solved.

Optionally, as shown in FIG. 2, the blood glucose monitoring device further comprises a display module 60 for displaying the blood glucose concentration values obtained by the control processing module 40.

The display module 60 herein may be a display of any type, for example, a Liquid Crystal Display (LCD), an Organic Light-Emitting Diode (OLED) display, or the like.

Further, the display module 60 may also have a touch function. On the basis of this, in order to enable the blood glucose monitoring device to meet different user demands, in addition to displaying the blood glucose concentration value on the display interface of the display, an identifier of a button for turning off the micro-current stimulation module 20 may also be displayed on the display interface of the display under the control of the control processing module 40. Normally, the micro-current stimulation module 20 may be turned on by default, so that when the blood glucose monitoring device is used, the control processing module 40 controls the micro-current stimulation module 20 to work. When the user does not need the stimulus from the micro-current stimulation module 20, the user may touch to select the identifier of the button for turning off the micro-current stimulation module 20 on the display interface, so that the control processing module 40 does not control the micro-current stimulation module 20 to work.

In addition, under the control of the control processing module 40, an identifier for selecting whether the display module 60 should work or whether the wireless communication module 50 should work may also be displayed on the display interface of the display so as to improve user experience.

It should be noted that the display surface of the display module 60 needs to be flush with the housing 10 or protrude from the housing 10. The wireless communication module 50 may be disposed within the housing 10 or embedded in the housing 10.

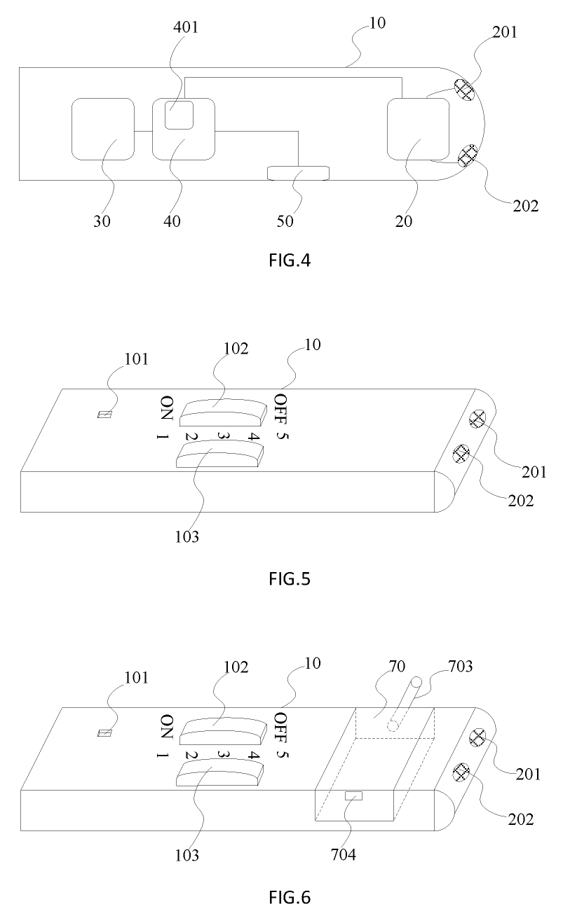

Optionally, as shown in FIG. 4, a current intensity control module 401 is integrated in the control processing module 40 for controlling intensity of the current generated by the micro-current stimulation module 20.

On this basis, as shown in FIG. 5, a current intensity regulation button 103 may be provided on the surface of the housing 10 for controlling the intensity of the current generated by the micro-current stimulation module 20 through the current intensity control module 401.

Here, several levels may be set for the current intensity, for example, as shown in FIG. 5, five levels may be set, and said levels are corresponding to a plurality of current intensities in a one-to-one manner. As the level rises, the current intensity gradually increases. Thus appropriate current intensities can be selected for stimulation according to the user's need, thereby improving user experience.

It shall be noted that the embodiment of the present disclosure is not limited to five levels, and the number of levels may be specifically set according to the actual situation.

Under the condition that the blood glucose monitoring device also comprises the display module 60 and the display module 60 has a touch function, the control processing module 40 can also be used to control the display module 60 to display the current intensity level and control the intensity of the current generated by the micro-current stimulation module 20 using the current intensity control module 401 according to the current intensity level selected by the user.

Similar to the function of the current intensity regulation button 103, the display module 60 may display, for example, five levels. When the user selects a corresponding level, the current intensity control module 401 can control the micro-current stimulation module 20 to generate a corresponding current intensity.

Optionally, as shown in FIG. 6, the blood glucose monitoring device further comprises a test paper box 70 detachably secured to the housing 10.

In the embodiment of the present disclosure, the test paper box 70 may be provided in the saliva glucose monitoring device so as to be easily carried. Besides, since the test paper box is detachable, when test paper in the test paper box runs out, the test paper box 70 can be easily replaced.

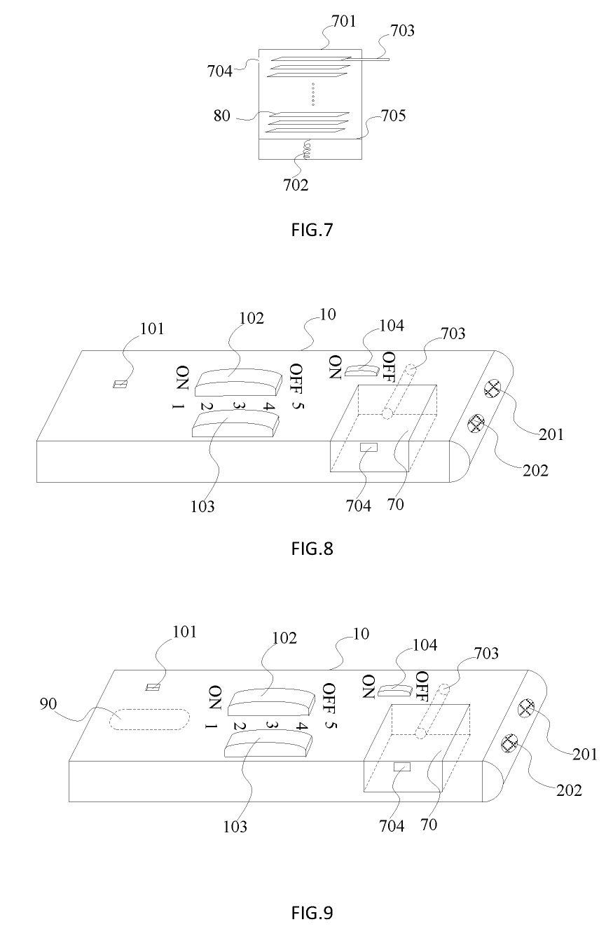

Further optionally, as shown in FIGS. 6-7, the test paper box 70 comprises a box body 701, an elastic component 702 provided at the bottom of the box body, and a push rod 703 provided at the top of the box body.

A test paper outlet 704 is provided on a side of the box body 701 at a position close to the top; the push rod 703 is disposed opposite to the test paper outlet 704, and the push rod 703 extends from inside of the box body to outside of the box body.

In the embodiment of the present disclosure, the test paper box is operated in such a manner as that the elastic component 702 is in a compressed state when the test paper box 70 is filled with test paper 80; by controlling the push rod 703, the uppermost test paper 80 in the box body 701 can be pushed out of the test paper outlet 704. In this process, since the test paper 80 above the elastic component 702 is reduced, the pressure received by the elastic component 702 is reduced, so the elastic component 702 gradually extends to push the test paper 80 next to the uppermost one to the top of the box body 701 so as to push it out from the test paper outlet 704 by the push rod 703. Subsequent test paper 80 is pushed out in a similar manner.

The elastic component 702 may be, for example, a spring. In order to prevent the test paper 80 from being damaged when coming into contact with the elastic component 702, a bearing tray 705 may be fixed above the elastic component 702.

It is to be noted that the test paper outlet 704 needs to be positioned in such a manner as to expose the test paper outlet 704 on the housing 10 after loading the test paper box 70 into the blood glucose monitoring device. Thus, by controlling the push rod 703, the test paper 80 can be automatically pushed out.

Optionally, the portion of the push rod 703 extending to the outside of the box body 701 can be located within the housing 10 and connected to the control processing module 40. On this basis, optionally, as shown in FIG. 8, a test paper control button 104 is provided on the surface of the housing 10 for controlling the push rod 703 to push the test paper 80 out of the test paper box 70 through the control processing module 40.

As shown in FIG. 8, when the test paper control button 104 is switched from the OFF state to the ON state, the control processing module 40 can control the push rod 703 to push a test paper 80 out of the test paper box 70, then the test paper control button 104 is switched from the ON state to the OFF state. As long as the control processing module 40 detects that the test paper control button 104 is switched from the OFF state to the ON state, the push rod 703 can be controlled to push out a test paper 80. This improves user experience significantly.

Of course, under the condition that the blood glucose monitoring device also comprises the display module 60 and the display module 60 has a touch function, the control processing module 40 can also be used to control the display module 60 to display the test paper control button, and, in accordance with the user's selection, control the push rod 703 to push out the test paper 80.

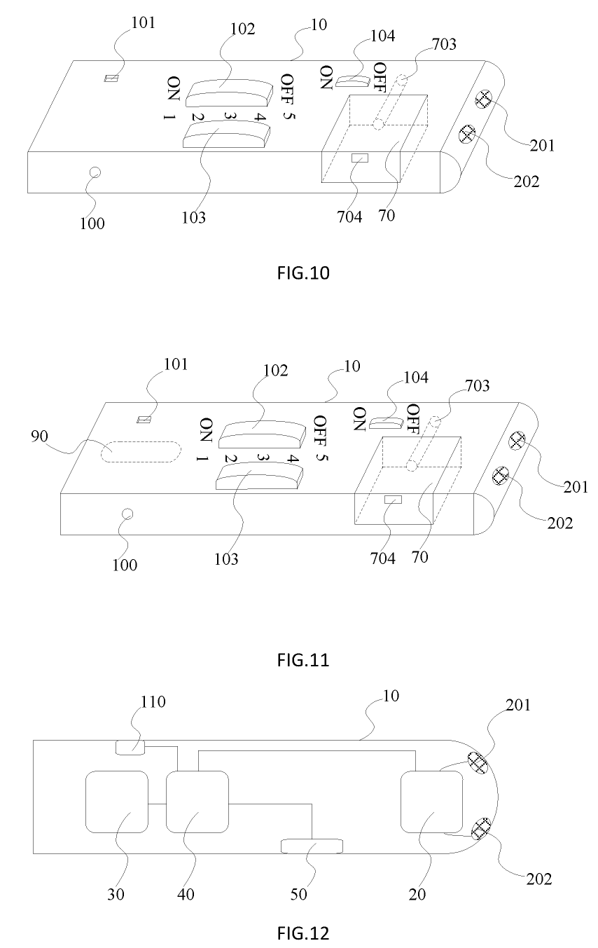

In order to realize normal operation of the blood glucose monitoring device, power must be supplied to respective modules, so a power supply module (not shown in the figure) is need in the blood glucose monitoring device. On this basis, as shown in FIG. 9, a battery placement area 90 is provided in the housing 10 for placing a battery for supplying power to the power supply module. Optionally, as shown in FIG. 10, a power supply interface 100 is provided on the housing 10, which can be connected to an external power supply for supplying power to the power supply module. Optionally, as shown in FIG. 11, not only the battery placement area 90 is provided in the housing 10, but also the power supply interface 100 is provided on the housing 10.

Optionally, as shown in FIG. 12, the blood glucose monitoring device further comprises an alarm module 110 for providing an alarm signal under the condition that the control processing module 40 determines that the blood glucose concentration value is greater than the preset threshold value.

Here, the alarm module 110 may be a device having an alarm function such as a buzzer, a light emitting diode, or the like. The alarm module 110 may be disposed within the housing 10, or embedded in the housing 10, or disposed on the outer surface of the housing 10.

In the embodiment of the present disclosure, when the blood glucose concentration value is relatively high, the user can know his/her physical condition in time.

Optionally, the housing 10 is made of a flexible material. Thus when the blood glucose monitoring device contacts the user's skin to stimulate the user's parotid gland to produce fresh saliva, the user will not get uncomfortable because of the material of the housing 10.

Optionally, the material of the housing 10 may be silica gel. This is because silica gel is soft and will not irritate the user's skin.

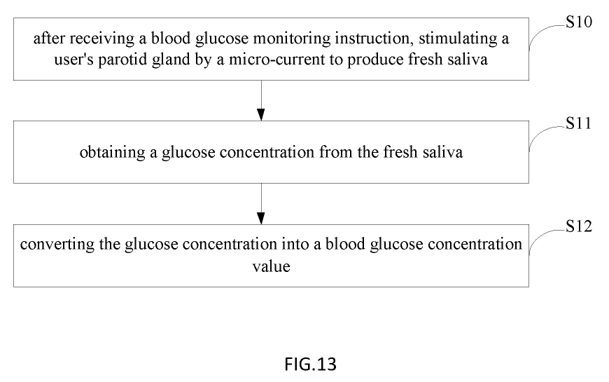

An embodiment of the present disclosure further provides a monitoring method for a blood glucose monitoring device, as shown in FIG. 13, which comprises the steps of:

S10: after receiving a blood glucose monitoring instruction, stimulating a user's parotid gland by a micro-current to produce fresh saliva.

Here, the instruction can be controlled by the user. Take the above-described blood glucose monitoring device as an example, a power switch may be provided on the blood glucose monitoring device, and when the user controls to turn on the power switch, it means that the control processing module 40 receives the blood glucose monitoring instruction.

The micro-current stimulation module 20 may generate a pulse current, or it may generate a constant current. The intensity of the micro-current may be at a level of microampere (μA), for example, within a range of 1-500 μA.

When the blood glucose monitoring device of the present disclosure is used, the two electrodes, i.e., the first electrode 201 and the second electrode 202, can be brought into contact with the skin at the parotid gland, and the micro-current generated by the micro-current stimulation module 20 stimulates the parotid gland to produce fresh saliva.

S11: obtaining a glucose concentration from the fresh saliva.

Still take the above-described blood glucose monitoring device as an example, the glucose concentration in the saliva can be acquired by the saliva glucose acquisition module 30, wherein the glucose concentration can be represented by current or be a glucose concentration value converted from the current value.

S12: converting the glucose concentration into a blood glucose concentration value. The control processing module 40 may be used to converting the glucose concentration into the blood glucose concentration value. The blood glucose concentration y has a linear relationship with the glucose concentration x. In addition, the blood glucose concentration value can be sent to an external device for easy management and storage.

By means of the monitoring method provided in embodiments of the present disclosure, the user's parotid gland can be stimulated by micro-current to produce fresh saliva, so influences from food residues or other substances in the mouth can be eliminated, and influence to the accuracy of measurement by the oral environment can be reduced, thereby improving monitoring accuracy and reliability, meanwhile, the problem that the test cannot be carried out when the user has a dry mouth without saliva is also solved.

Optionally, said monitoring method further comprises: displaying the blood glucose concentration value.

For example, in the above-described blood glucose monitoring device, the control processing module 40 controls the display module 60 to display the blood glucose concentration value.

Optionally, said monitoring method further comprises: sending an alarm when the blood glucose concentration value is greater than the preset threshold value.

For example, in the above-described blood glucose monitoring device, when the control processing module 40 determines that the blood glucose concentration value is greater than the preset threshold value, it can control the alarm module 110 to provide an alarm signal.

It will be understood by those of ordinary skill in the art that all or some of the steps for implementing the method embodiments described above may be carried out by hardware associated with program instructions, and the program may be stored in a computer-readable storage medium to carry out steps of the method embodiments described above when executed. The aforementioned storage medium includes a variety of mediums, such as ROM, RAM, disk, or optical disk, that can store program codes.

The above described are only exemplary embodiments of the present disclosure, which do not intend to limit the protection scope of the present disclosure. Any variation or substitution that is easily conceivable by those skilled in the art within the technical scope disclosed by the present disclosure shall fall into the protection scope of the present disclosure. Thus the protection scope of the present disclosure is defined by the appended claims.

1. A blood glucose monitoring device, comprising:
a housing;
a micro-current stimulation module connected to two electrodes and generating a current, the two electrodes being located on a surface of the housing and the current stimulating the parotid gland of a user through the two electrodes to produce fresh saliva;
a saliva glucose acquisition module configured to acquire a glucose concentration in the saliva;
a control processing module configured to control the micro-current stimulation module to work and to obtain a blood glucose concentration value from the glucose concentration.

2. The blood glucose monitoring device according to claim 1, further comprising a display module configured to display the blood glucose concentration value obtained by the control processing module.

3. The blood glucose monitoring device according to claim 1, further comprising a wireless communication module through which the control processing module communicates with an external terminal.

4. The blood glucose monitoring device according to claim 1, wherein a current intensity control module is integrated in the control processing module to control intensity of current generated by the micro-current stimulation module.

5. The blood glucose monitoring device according to claim 4, further comprising a current intensity regulation button operated to control current intensity of the micro-current stimulation module through the current intensity control module.

6. The blood glucose monitoring device according to claim 1, further comprising a test paper box detachably secured to the housing.

7. The blood glucose monitoring device according to claim 6, wherein the test paper box comprises a box body, an elastic component provided at the bottom of the box body and a push rod provided at the top of the box body;
wherein a test paper outlet is provided on a side of the box body at a position close to the top;
the push rod is disposed opposite to the test paper outlet, and the push rod extends from inside of the box body to outside of the box body.

8. The blood glucose monitoring device according to claim 7, wherein a portion of the push rod extending to the outside of the box body is located within the housing and is connected to the control processing module; and wherein the blood glucose monitoring device further comprises a test paper control button operated to control the push rod by the control processing module so as to push the test paper out of the test paper box.

9. The blood glucose monitoring device according to claim 1, further comprising a power supply module for supplying power to the blood glucose monitoring device.

10. The blood glucose monitoring device according to claim 9, wherein a battery placement area is provided in the housing to place a battery for supplying power to the power supply module.

11. The blood glucose monitoring device according to claim 9, wherein a power supply interface is also provided on the housing, through which an external power supply supplies power to the power supply module.

12. The blood glucose monitoring device according to claim 1, further comprising an alarm module configured to provide an alarm signal under the condition that the control processing module determines that the blood glucose concentration value is greater than a preset threshold value.

13. The blood glucose monitoring device according to claim 1, wherein the housing is made of a flexible material.

14. A monitoring method for the blood glucose monitoring device, comprising:
after receiving a blood glucose monitoring instruction, stimulating a user's parotid gland by a micro-current to produce fresh saliva;
obtaining a glucose concentration from the fresh saliva;
converting the glucose concentration into a blood glucose concentration value.

15. The monitoring method according to claim 14, further comprising displaying the blood glucose concentration value.

16. The monitoring method according to claim 14, further comprising sending an alarm signal when the blood glucose concentration value is greater than a preset threshold value.

17. The blood glucose monitoring device according to claim 10, wherein a power supply interface is also provided on the housing, through which an external power supply supplies power to the power supply module.
